(19) United States
(12) Patent Application Publication
(10) Pub. No.: US 2026/0127256 A1
(43) Pub. Date: May 7, 2026

(54) USER AUTHENTICATION FOR A DRUG DELIVERY DEVICE

(71) Applicant: Sanofi, Paris (FR)

(72) Inventor: Michael Helmer, Frankfurt am Main (DE)

(21) Appl. No.: 18/705,693

(22) PCT Filed: Nov. 2, 2022
(86) PCT No.: PCT/EP2022/080594

§ 371 Date: Apr. 29, 2024

(30) Foreign Application Priority Data
Nov. 3, 2021 (EP) 21315204.4

(51) Int. Cl. G06F 21/31; A61M 5/20

(52) U.S. Cl. CPC G06F 21/316; A61M 5/20; A61M 2005/2026; A61M 2005/2073; A61M 2205/3546; A61M 2205/6009; A61M 2230/63

(57) ABSTRACT

A method of authenticating a user for using a drug delivery device includes acquiring motion data of the user; comparing acquired motion data with stored motion data of a user-specific data record and generating a data matching index being indicative of a degree of matching between the acquired motion data and the stored motion data; and enabling, disabling, triggering, or terminating an execution of a function of the drug delivery device based on the data matching index.

### CROSS REFERENCE TO RELATED APPLICATIONS

The present application is the national stage entry of International Patent Application No. PCT/EP2022/080594, filed on Nov. 2, 2022, and claims priority to EP Application No. 21315204.4, filed on Nov. 3, 2021, the disclosures of which are incorporated herein by reference.

### TECHNICAL FIELD

The present disclosure relates to a method of authenticating a user to use a drug delivery device, to an authentication system to authenticate the user, to a computer program for user authentication, and to a mobile electronic device for providing user authentication.

### BACKGROUND

Drug delivery devices for setting and dispensing a single or multiple doses of a liquid medicament are as such well-known in the art. Generally, such devices have substantially a similar purpose as that of an ordinary syringe.

### SUMMARY

Drug delivery devices, e.g. injection devices and needle based injection system (NIS) devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Needle based injection to be conducted with a variety of injection devices is more and more associated with measuring and logging data regarding the amount of medicament set and set or dispensed or injected at a particular date or time. For this, injection devices may be provided with an electronic circuit providing data acquisition and/or data logging as well as communication of collected or measured dispensing related data.

Such electronic circuits or electronic units may be either implemented or integrated into the injection device itself. Alternatively, such electronic circuits or electronic units may be provided by a supplementary device or an add-on device configured for releasable attachment to the injection device and being operable to log injection-related data during use of the injection device. Moreover, electronic circuits for data logging may be also provided with a communication interface so as to transmit previously stored injection-related data to an external electronic device for further data processing.

Some users or patients may need to administer different medicaments by using different drug delivery devices, where the devices may, at first glance, look similar. Also, devices may be kept in households, where not only a single but a plurality of persons is in theory able to access the device.

It is therefore desirable to provide an improved method and a system to authenticate a user to use a particular drug delivery device. It is further desirable to provide a computer program and to provide respective hardware components to enhance patient safety and to prevent unauthorized use of a drug delivery device by a user not authorized to make use of the drug delivery device.

### SUMMARY

According to one aspect the present disclosure relates to a method of authenticating a user to use a drug delivery device, such as an injection device. The method includes the steps of acquiring of motion data of the user and comparing the acquired motion data with stored motion data of a user specific data record. Based on this comparison there is generated a data matching index. The data matching index is indicative of a degree of matching or similarity between the acquired motion data and the stored motion data. Furthermore, a function, e.g. an electronically implemented function of the drug delivery device is either enabled, disabled, triggered or terminated on the basis of the data matching index.

Typically, a user authorized to use the drug delivery device is identified by its motion data. The motion data is typically acquired or collected either by the drug delivery device itself or by a mobile electronic device, e.g. carried along with the user or attached to the user. Typically, motion data of the user is acquired over a predefined period of time and/or motion data is acquired at specific and predefined points of time or at particular days. The motion data is characteristic and unequivocal for each user using a drug delivery device. It may provide a kind of a fingerprint of each user.

Typically, a first user is characterized by motion data of a first user specific data record. Accordingly, a second user is characterized by motion data of a second user specific data record. By comparing motion data that has been acquired while a user being in motion or while a user executes or conducts a particular movement or motion pattern and by comparing the acquired motion data of this user with motion data of a user specific data record a user of a mobile electronic device and/or a user of a drug delivery device can be identified.

Typically, and with some examples the user specific data record is assigned or mapped to a particular drug delivery device. When the same user makes use of the drug delivery device and when the motion data of this particular user is acquired a comparison of the acquired motion data with the user specific data record assigned or mapped to the drug delivery device shows a high degree of matching or similarity. Accordingly, the respective user will be authorized to use the drug delivery device.

Accordingly, at least one function of the drug delivery device is enabled, disabled, triggered or terminated as demanded by the user of the drug delivery device. In other situations and when a second user, which is not authorized to use the drug delivery device, intends to use this particular device, the motion data of this second user is collected or acquired and is compared with motion data of the user specific data record of the first user, which is mapped to this particular drug delivery device. Since motion data of the second user distinguishes from the motion data of the user specific data record there will be determined a rather low degree of matching or similarity between the acquired motion data of the second user and the stored motion data of the user specific data record of the first user. Accordingly, the respective function of the drug delivery device will be enabled, disabled, triggered or terminated on the basis of the data matching index.

With some examples and when a user is authenticated to use the drug delivery device the respective device function may be enabled or triggered. In other situations and when analysis or evaluation of the motion data of the user reveals that the user is not authenticated to use the drug delivery device, e.g. due to a rather low degree of matching between the acquired motion data and the stored motion data, the respective function of the drug delivery device may be disabled or terminated.

According to a further example the motion data of the user is acquired by an electronic circuit including at least one of a communication interface and a sensor. With some examples the electronic circuit is implemented or integrated into the drug delivery device or into a supplementary device attachable to the drug delivery device. With other examples the electronic circuit is implemented or integrated into a mobile electronic device, such as a smart phone, a smart watch, a tablet computer, a fitness tracker or a wireless tag. Typically, such mobile electronic devices may be carried along with the user anyway. This way, the respective mobile electronic device may occasionally or permanently collect or acquire motion data of a user. It may be hence operable to conduct the comparison of the acquired motion data with stored motion data of the user-specific data record.

With other examples and when the electronic circuit is implemented in or is attached to the drug delivery device a movement of the drug delivery device, e.g. conducted by a user of the device can be detected, e.g. when the user grips the injection device or takes the injection device from a desk.

The sensor may recognize a movement or movement pattern and/or gestures of a user when moving himself or when moving at least one of a mobile electronic device and the drug delivery device. This way, a movement profile or pattern of a user or typical gestures of a user can be detected and/or quantitatively measured. Such movement patterns or gestures can be permanently or occasionally acquired and can be compared with movement or motion patterns and/or gesture data stored in one of the drug delivery device, the supplementary device and the mobile electronic device.

Deviations of acquired motion data from stored motion data can be detected, thus leading to a rather low degree of matching and to the generation of a rather low matching index. Depending on the size of the matching index execution of the function of the drug delivery device, which typically requires a respective authorization can be enabled, disabled, triggered or terminated.

According to a further example at least a portion of the motion data of the user is acquired by the sensor. The sensor includes at least one of an acceleration sensor, a rotation sensor, a position sensor, a distance sensor and a physiologic data capturing sensor. The sensor is operable to generate electrical sensor signals. The sensor is typically operatively connected to a processor operable to evaluate and/or to analyze the sensor signal so as to derive and/or to characterize motion data being indicative of characteristic motions or gestures of the user when moving the electronic circuit and hence the sensor.

With some examples the sensor includes a physiologic data capturing sensor. Such a sensor may be operable to measure at least one of a body temperature, blood pressure, heartbeat rate, breathing rate, oxygen saturation or other physiologic parameters being indicative of the well-being of the user.

When the sensor includes a position sensor it may be operable to determine an absolute or relative position, e.g. by way of communicating with a satellite-based positioning system or by communicating with another electronic device. This way, either an absolute position of the sensor, e.g. in form of geographic coordinates or e.g. in form of relative coordinates in relation to another electronic device can be measured or determined.

With further examples the electronic circuit is also equipped with a clock by way of which the motion data or position data acquired by the sensor can be mapped to a date, to a point of time and/or to a time interval. This way, a motion profile or movement profile over time can be generated being indicative of a typical motion pattern of a user.

As an example, a user may be identified to be at a certain geographic position at a typical point of time or time interval. For instance, a first user may be characterized by staying 8 hours a day, e.g. from 9 to 17 o'clock in a particular office building. A user may leave the office building for a lunch break at a regular time and for a time interval of e.g. half an hour.

Moreover, a user may be recognized by moving from home to work e.g. by car or by public transportation at a particular time of the day. Such motion data may be captured and acquired over a comparatively long time interval. When motion data currently acquired should strongly deviate from the long-term average of respective motion data this may be indicative of a situation, in which a different user uses the respective electronic circuit.

Deviations of acquired motion data from stored motion data or average motion data may be quantitatively expressed by the data matching index. This way, there can be generated a probability or a likelihood if and in how far motion data currently acquired is obtained from a particular user mapped or assigned to the user specific data record.

According to a further example at least a portion of the motion data of the user is acquired by the communication interface through wireless communication with at least one of a satellite-based positioning system, an access point of a communication network, a counterpart communication interface of a mobile electronic device, such as a supplementary device attachable to the drug delivery device, an electronic unit or electronic circuit of the drug delivery device or a mobile electronic device.

This way, there is provided a high degree of flexibility of how to obtain motion data of a user. Insofar, motion data of a user can be obtained even by way of an electronic device being void of a sensor, such as an acceleration sensor, a rotation sensor, a position sensor and a distance sensor. For instance in an urban area there may be provided numerous access points of a communication network to which the communication interface of the electronic circuit establishes a communication link as the electronic circuit is subject to a movement or motion.

Simply by logging establishing of communication links with different access points of a communication network during a movement or motion from one place to another there can be generated a motion pattern or motion profile which may be indicative of a habitual movement or motion of the user.

Of course, the communication interface may establish a communication link, e.g. a downlink communication with a satellite-based positioning system. This way, the communication interface may be operable to determine absolute or global geographic coordinates or a respective geographic position. The communication interface may also communicate with counterpart communication interfaces of further mobile or stationary electronic devices. Also here, at least a relative distance or a relative position information between the communication interface of the electronic circuit and the counterpart communication interface of the further mobile or stationary electronic device can be obtained.

Since there are many different ways of how to acquire motion data of a user the method of authenticating the user by way of the user's motion data can be implemented with a large variety of different hardware and software configurations.

According to a further example the motion data, which is either acquired or which is stored includes at least one of gesture data being indicative of characteristic gestures of the user and a motion pattern being indicative of at least one of a position and a motion profile of the user at a specific point of time or during a specific time interval.

Mutually mapping a position or movement with a point or time or with time interval is characteristic for a user of a drug delivery device. This way and by determining a position and/or motion of a user at a specific point of time or during a specific time interval the user can be precisely identified. The gesture data may be typically acquired by the sensor of the electronic circuit. Gesture data may characterize the way of how a person or user moves or which types of gestures are typical for such a user.

Based on signals of the sensor there may be determined if a left-handed user or if a right-handed user moves the electronic circuit, e.g. by lifting or by taking at least one of the electronic circuit and the drug delivery device equipped with the electronic circuit. A typical gesture may be defined by an acceleration or velocity profile being indicative of how a particular object is taken by a user and/or at which velocity such a procedure takes place. Of course, this requires that the respective electronic circuit equipped with the sensor is attached to e.g. a hand or wrist of the user.

According to a further example the user-specific data record is obtained by at least one of deriving and storing motion data from previously acquired motion data of the user or by requesting and receiving at least one of a user specific motion pattern and user specific gesture data from a database. With some examples the method is implemented as a self-learning method. For instance, motion data of the user that has been acquired can be stored as user specific motion data in the user specific data record. A repeated acquisition of motion data, e.g. captured at the same point of time or at a same or at a similar place or position may then be also stored in the user specific data record.

Before or upon storing acquired motion data in the user specific data record the acquired motion data may be evaluated and may be mapped to a characteristic motion pattern or gesture of the user. Repeatedly acquired motion data and in particular repeatedly acquired motion data of one and the same gesture or motion pattern may enrich the user specific data record.

For instance and with a rather simple example a motion data can e.g. being characteristic of a user leaving home in order to get to work and the respective movement can be mapped to a particular point or period of time. With a long-term acquisition of motion data it may be determined that a user typically leaves his home between 7:15 and 7:25 from Monday to Friday.

In case that motion data currently acquired reveals that the user leaves home e.g. at 9 o'clock in the morning on a Tuesday this may be a direct indication of a rather high degree of mismatch between the acquired motion data and their stored motion data.

Accordingly, this mismatch may be used to deauthorized the user to use the drug delivery device or at least to prompt the user to verify if he is really authenticated to use the drug delivery device.

With other examples a user specific motion pattern may be programmed or may be defined by the user himself. With further examples a user specific motion pattern and/or user specific gesture data may be taken from a database. Nowadays there exist numerous service providers, e.g. a manufacturers or distributors of e.g. fitness trackers that collect at least one of regular motion data, motion patterns, motion profiles or physiologic data of a user carrying the fitness tracker. Here, respective user specific motion data or user specific physiologic data may be stored in a database.

The method may now directly request access to such a database in order to obtain or to evaluate this type of user specific data for the purpose of verifying user authentication or user authorization. Here, it is not necessary that the drug delivery device or the electronic circuit of the drug delivery device permanently collects or acquires motion data of the user. It may be sufficient when the respective data, which is collected anyway by some other entity is made available through communication via a network.

According to another example execution of the function of the drug delivery device is enabled or triggered when the data matching index is above an upper data matching threshold. With some examples the function of the drug delivery device may be setting of a dose or dispensing of a dose of a medicament. The setting or dispensing of a dose may be enabled or triggered only when the data matching index is above an upper data matching threshold and hence when the user of the drug delivery device has been identified by his motion data. This way, patient safety and unintended cross use of a drug delivery device can be prevented.

With a further example execution of the function of the drug delivery device is disabled or terminated when the data matching index is below a lower data matching threshold. Here, and with the example of a dose setting or dose dispensing function of the drug delivery device dose setting or dose dispensing may be effectively impeded as long as the data matching index is below the lower data matching threshold. A user exhibiting motion data that does not match with stored motion data of the user specific data record will not be allowed to execute the particular function of the drug delivery device, such as setting and/or dispensing of a dose of the medicament.

With other examples and depending on the function of the drug delivery device it is also conceivable, that execution of the function is actually triggered when the data matching index is below the upper data matching threshold and is above the lower data matching threshold. Here and as a typical example the function of the drug delivery device may include or include generation of a signal or of an alert to the user to manually check his authorization to use the drug delivery device.

In such situations at least one of the mobile electronic device, the drug delivery device or a supplementary device may be configured to generate a signal or indication to the user to pay attention of not using a wrong drug delivery device.

With some examples there may be autonomously triggered a separate authentication routine prompting the user to authenticate with the drug delivery device by e.g. entering an identification code or the like identifier.

According to a further example the method further includes the step of entering into a dialogue with the user or generating and outputting a user request when the data matching index is below the upper data matching threshold and above the lower data matching threshold.

As a further step the function of the drug delivery device may be then enabled or triggered in response to the user dialogue or in response to a reaction of the user to the user request. Here, a user may be prompted to confirm that he is in fact authorized to use the drug deal delivery device. Upon confirmation the respective device function may be enabled or may be directly triggered.

If a user fails to respond appropriately the in a user dialogue or upon user request the respective function of the drug delivery device may be disabled or ay remain disabled or a function currently executed may be promptly terminated.

In a further aspect the present disclosure relates to an authentication system. The authentication system is configured and/or operable to authenticate and/or to authorize a user to use a drug delivery device. The authentication system includes an electronic circuit integrated into or attachable to the drug delivery device. The electronic circuit is operable to enable, disable, to trigger or to terminate execution of a function of the drug delivery device. The function is typically implemented as an electronic function of the drug delivery device.

The authentication system further includes at least one of a sensor and a communication interface operable to acquire motion data of the user intending to use the drug delivery device. The authentication system further includes a storage operable to store motion data of a user specific data record. The authentication system further includes at least one processor operable to compare acquired motion data with stored motion data. The processor is further operable to generate a data matching index being indicative of a degree of matching between the acquired motion data and the stored user-specific motion data. The data matching index is configured to effectuate at least one of an enabling, disabling, triggering or terminating execution of the function of the drug delivery device.

Typically, the authentication system is implemented to execute the above-mentioned method of authenticating a user to use a drug delivery device. Insofar all features, effects and benefits as described above in connection with the method of authenticating and/or authorizing a user equally apply to the authentication system; and vice versa.

The user specific data record contains stored motion data being characteristic of typical motions, gestures or of a motion or gesture profile of the user. The sensor and/or the communication interface being operable to acquire motion data may be implemented either in the drug delivery device, in a supplementary device attachable to the drug delivery device or in a mobile electronic device. Any of such devices is typically moved by the user or is carried along with the user. This way, at least one of the sensor and the communication interface is operable to acquire, to collect, to track and/or to log motion data of the user.

The acquired motion data of the user, which typically includes at least one of gesture data and a motion pattern of the user may be categorized and may be compared with stored motion data of the user specific data record. The comparison of acquired and stored motion data may be conducted on the basis of a predefined timeline and/or on the basis of an absolute or relative position of the at least one sensor and the communication interface, e.g. in relation to another electronic device.

With some examples motion data may be also required by requesting and receiving a motion pattern or motion profile from a database. In a typical scenario of use a motion pattern or motion profile of a user equipped with a smartphone or smartwatch can be tracked and/or logged by the respective mobile electronic device. The motion data and/or the motion profile as well as typical gestures can be uploaded in a database and can be requested and downloaded upon request of the authentication system for the purpose of user recognition.

Depending on the degree of matching and hence depending on the size of the data matching index the processor may invoke data matching-specific and hence different actions with regard to the execution of the function of the drug delivery device. For instance, and with a rather high data matching index particular functions of the drug delivery device may be enabled or may be directly triggered. Such functions may include setting of a dose, dispensing of a dose, unlocking of the drug delivery device, initiating or conducting a user dialogue with the user, providing information to the user with regards to a use of the drug delivery device, and many others.

With a medium-sized data matching index some functions of the drug delivery device may be disabled or execution of a particular function of the drug delivery device may be terminated. For instance, with a medium-sized data matching index and hence with a medium degree of matching between the acquired motion data and the stored user-specific motion data, setting and/or dispensing of a dose may be deactivated and may be hence disabled. A mechanical or electromechanical interlock of the drug delivery device may be enabled or activated. At the same time execution of a dialogue with the user may be triggered so as to prompt the user to confirm if he is really entitled to use the drug delivery device.

With other scenarios of use a medium-sized data matching index may invoke a further user authentication routine. Here, the user may be prompted to enter a user specific or device specific authentication code in order to enable and/or to trigger the desired function of the drug delivery device.

With a rather low degree of matching between the acquired motion data and the stored user-specific data other functions of the drug delivery device may be enabled or disabled. Here, it is conceivable, that an electromechanical interlock of the drug delivery device is activated and that the user has to conduct an alternative routine or procedure to unlock the drug delivery device.

According to a further example the electronic circuit is part of a supplementary device attachable to the drug delivery device. Alternatively, the electronic circuit is part of an electronic unit integrated into the drug delivery device. Typically, the electronic unit includes a unit processor operable to execute the function of the drug delivery device. It may be then another or the same processor that is operable to compare acquired motion data with stored motion data and to generate or to derive the data matching index as well as to decide which function of the drug delivery device is enabled, disabled, triggered or terminated on the basis of the data matching index.

With other examples the electronic circuit is provided with a unit communication interface by way of which the electronic circuit receives at least one control command from another electronic device, wherein the other electronic device includes the processor operable to compare acquired motion data with stored motion data and being further operable to generate the data matching index.

With other examples the electronic circuit being part of the supplementary device or being part of the electronic unit integrated into the drug delivery device may operate standalone. The electronic circuit may acquire motion data of the user, e.g. when a user takes the drug delivery device from a desk. Here, the electronic circuit may be equipped with the sensor, e.g. implemented as one of an acceleration sensor, a rotation sensor, a position sensor, a distance sensor and/or a physiologic data capturing sensor. Then, the electronic circuit of the supplementary device or of the drug delivery device is able to acquire the motion data of the user. The same electronic circuit may be also configured to compare the acquired motion data with stored motion data. Here, the stored motion data may be stored locally in the electronic circuit of the supplementary device or in the electronic unit of the drug delivery device.

Alternatively, the electronic circuit is equipped with a communication interface so as to communicate with other electronic devices or with a network so as to request and to receive a user-specific motion pattern from a database.

According to a further example the authentication system includes at least one mobile electronic device. The at least one mobile electronic device includes at least one of the sensor and the communication interface to acquire at least a portion of the motion data of the user. With some examples it is the mobile electronic device that is operable to store the user-specific data record.

With further examples it is the mobile electronic device that includes the at least one processor. This way, it is the mobile electronic device that is operable to conduct the comparison of the actually acquired motion data with previously stored and hence user-specific motion data. It is then the mobile electronic device that generates the data matching index.

Accordingly, the mobile electronic device may be also configured to make a decision with regards to enabling, disabling, triggering or terminating execution of a particular function of the drug delivery device on the basis of the data matching index. Here, at least one or several of the above-described method steps of acquiring motion data, comparing of the acquired motion data as well as generating the data matching index and making a decision with regards to an enabling, disabling, triggering or terminating execution of the function of the drug delivery device is provided by the mobile electronic device.

The mobile electronic device may be implemented as a smartphone, as a smart watch, as a tablet computer, as a fitness tracker or as a wireless tag, typically carried along with the user intending to use the drug delivery device.

According to a further sample of the authentication system the electronic circuit includes a circuit communication interface configured to wirelessly communicate with the communication interface of the at least one mobile electronic device. With this example the electronic circuit is typically implemented with one of the supplementary device and the drug delivery device. By way of the circuit communication interface there may be established a communication link between the electronic circuit of one of the supplementary device and the drug delivery device with the at least one mobile electronic device. In this way, the electronic circuit implemented with one of the supplementary device and the drug delivery device and operable to effectuate at least one of enabling, disabling, triggering or terminating execution of the function of the drug delivery device can be controlled by the mobile electronic device.

With this example it may be the mobile electronic device that acquires motion data of the user. By way of the communication link between the electronic circuit and the at least one mobile electronic device acquired motion data can be transferred and transmitted from the mobile electronic device to the electronic circuit of at least one of the drug delivery device and the supplementary device. Further processing of the motion data may be then conducted onboard by the supplementary device or by the drug delivery device.

With other examples it is conceivable, that processing of the motion data, i.e. comparing of the acquired motion data with stored user-specific motion data is conducted by the mobile electronic device. The data matching index derived on the basis of this comparison may be generated by the mobile electronic device and may be transmitted to the electronic circuit of at least one of the supplementary device and the drug delivery device for enabling, disabling, triggering or terminating execution of the particular device function.

With further examples a decision-making with regard to an enabling, disabling, triggering or terminating execution of the function of the drug delivery device may be even conducted by the at least one mobile electronic device. A respective execution command generated by the mobile electronic device may be simply transmitted via the communication interface of the at least one mobile electronic device to the communication interface of the electronic circuit of one of the drug delivery device and the supplementary device. Upon receiving of such an execution command the electronic circuit of at least one of the supplementary device and the electronic unit of the drug delivery device may be then operable to enable, disable, trigger or terminate execution of the function of the drug delivery device accordingly.

With further examples it is the circuit communication interface that is operable to receive at least a portion of the motion data acquired by the at least one mobile electronic device. With other examples it is the mobile electronic device that includes at least one of an external electronic device, e.g. a smartphone, a wearable electronic device, e.g. a smartwatch or a fitness tracker, and a wireless tag. A wireless tag may be operable to establish a wireless communication link with another mobile electronic device or stationary electronic device.

At least one of an absolute or relative position of the wireless tag may be measured, determined and/or tracked by another electronic device so as to obtain motion data of a user that is equipped with the wireless tag. With typical scenarios of use a wireless communication link between the wireless tag and another electronic device as well as a communication link between at least one of the supplementary device and the drug delivery device with a mobile electronic device may be implemented on the basis of electromagnetic ultra-wideband technology.

According to a further example the processor of the authentication system is operable to enable, disable, trigger or to terminate execution of the function of the drug delivery device depending on the data matching index. This processor may be the same or another processor that compares the acquired motion data with stored motion data and which generates the data matching index.

With some examples there may be only one processor, e.g. of the supplementary device or of the drug delivery device that conducts the comparison of the acquired motion data with stored motion data. The same processor may be also operable to enable, disable, trigger or terminate execution of the function of the drug delivery device depending on the data matching index.

With other examples of the authentication system there may be provided numerous processors, e.g. implemented or integrated in at least one of the supplementary device and the drug delivery device and in one mobile electronic device. Here, a first processor may be operable to acquire motion data. The same or another processor may be operable to compare the acquired motion data with stored motion data of the user specific data record. A further processor may be operable to generate the data matching index and still another processor may be operable to process the data matching index so as to decide, which function of the drug delivery device is enabled, disabled, triggered or terminated on the basis of the data matching index.

The numerous processors that may be distributed among a plurality of hardware components, such as the supplementary device, the drug delivery device or any mobile electronic device are typically operable to communicate with each other, e.g. by establishing a respective wireless communication links by making use of respective wireless communication interfaces.

According to a further aspect the present disclosure relates to a computer program including computer readable instructions, which, when executed by at least one processor of an authentication system as described above causes the processor to acquire motion data of the user, to compare acquired motion data with stored motion data of a user-specific data record, to generate a data matching index being indicative of a degree of matching between the acquired motion data and the stored motion data and to enable, to disable, to trigger or to terminate execution of a function of the drug delivery device on the basis of the data matching index.

Typically, the computer program is configured to be executed by at least one or several hardware components of the authentication system as described above in order to execute the method of authenticating a user as described above. Insofar, all features, effects and benefits as described above in connection with the method of authenticating a user and with the authentication system equally apply to the computer program; and vice versa.

The computer program may be executed by numerous hardware components in a distributed hardware environment. Only some portions or the entirety of the computer readable instructions may be conducted by a processor of the electronic circuit of the supplementary device, of the drug delivery device, of a mobile electronic device or by a processor of a database being accessible via a data communication network. With some examples the computer program may be executed standalone, e.g. by a processor of an electronic circuit of the supplementary device or by the electronic unit of the drug delivery device. With some examples the computer readable instructions are at least partially or entirely executed by an electronic circuit and a processor of a mobile electronic device.

According to another aspect the present disclosure relates to a set including one of an arbitrarily selected plurality of, or all of the following:
a drug delivery device provided with an electronic circuit of the authentication system,
a supplementary device attachable to the drug delivery device and provided with an electronic circuit of the authentication system as described above, and
a mobile electronic device including at least one of the sensor and the communication interface of the authentication system as described above.

Generally, the scope of the present disclosure is defined by the content of the claims. The injection device is not limited to specific embodiments or examples but includes any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about 4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30 -N-myristoyl-ThrB29LysB30 human insulin; B30 -N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-carboxyheptadecanoyl)-des(B30) human insulin and B29-N-(w-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not include a full-length antibody polypeptide, but that still includes at least a portion of a full-length antibody polypeptide that is capable of binding to an antigen. Antibody fragments can include a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of a method of authenticating a user to use a drug delivery device as well as numerous hardware configurations including an authentication system, an electronic circuit, a drug delivery device, a supplementary device and numerous implementations of mobile electronic devices will be described in greater detail by making reference to the drawings, in which:

FIG. 1 illustrates an example of a drug delivery device implemented as a pen-type injection device,

FIG. 2 shows an injection device equipped with a supplementary device,

FIG. 3 shows a longitudinal cross-section through an example of a pen-type injection device,

FIG. 4 shows numerous hardware components of an authentication system,

FIG. 5 is illustrative of a block diagram of an example of the authentication system,

FIG. 6 is illustrative of a block diagram of another implementation of the authentication system,

FIG. 7 shows a block diagram of a further implementation of the authentication system,

FIG. 8 is illustrative of a block diagram, wherein a supplementary device attached to an injection device is operable to communicate with at least one or a plurality of mobile electronic devices,

FIG. 9 is illustrative of a wireless communication between a mobile electronic device and an electronic unit of the drug delivery device,

FIG. 10 schematically illustrates different users provided assigned with different data records,

FIG. 11 shows a flowchart of a method to authenticate a user to use a drug delivery device,

FIG. 12 is indicative of a comparison of acquired motion data with stored motion data,

FIG. 13 shows a flowchart of a further implementation of the method of authenticating a user,

FIG. 14 shows a further flowchart of the method of authenticating the user and

FIG. 15 shows a further flowchart of the method of user authentication.

### DETAILED DESCRIPTION

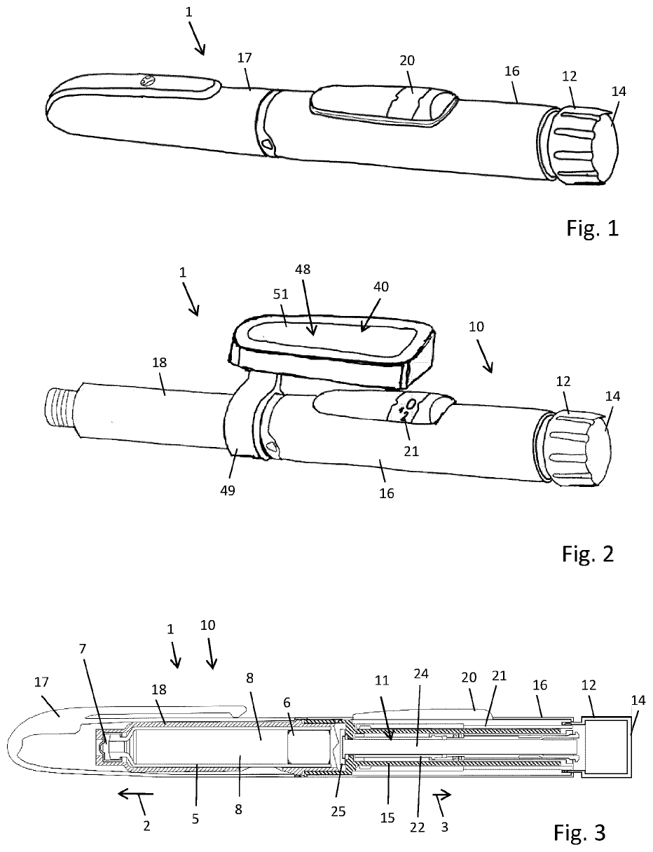

In FIGS. 1-3 an example of a drug delivery device 1 implemented as an injection device in is schematically illustrated. The drug delivery device 1 includes an elongated housing 16, e.g. of cylindrical or tubular shape. The housing 16 extends along a longitudinal direction. Towards a distal direction 2 and hence towards a distal end an injection needle 19 can be attached to a container housing 18 forming or constituting a distal part of the housing of the injection device 10. Towards a proximal direction 3 and hence at a proximal end of the injection device 10 there is provided a dose dial 12 and/or a dose button 14 allowing to set a dose and to dispense a dose of the medicament 8, respectively.

The container housing 18 is configured and shaped to accommodate a medicament container 5. The medicament container 5 may include a tubular-shaped barrel. The medicament container 5, e.g. implemented as a standard cartridge may be sealed towards the proximal direction 3 by way of a movable stopper 6. The distal end of the medicament container 5 is typically sealed by a pierceable seal 7. Hence, an outlet of the medicament container 5 is covered by the pierceable seal and is fixed to a head of the medicament container 5 e.g. by a crimped cap (not illustrated).

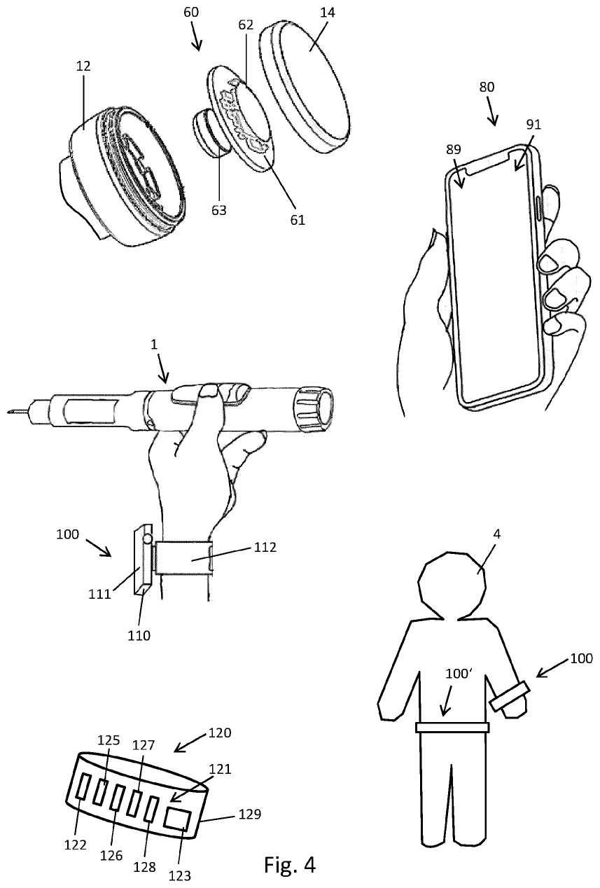

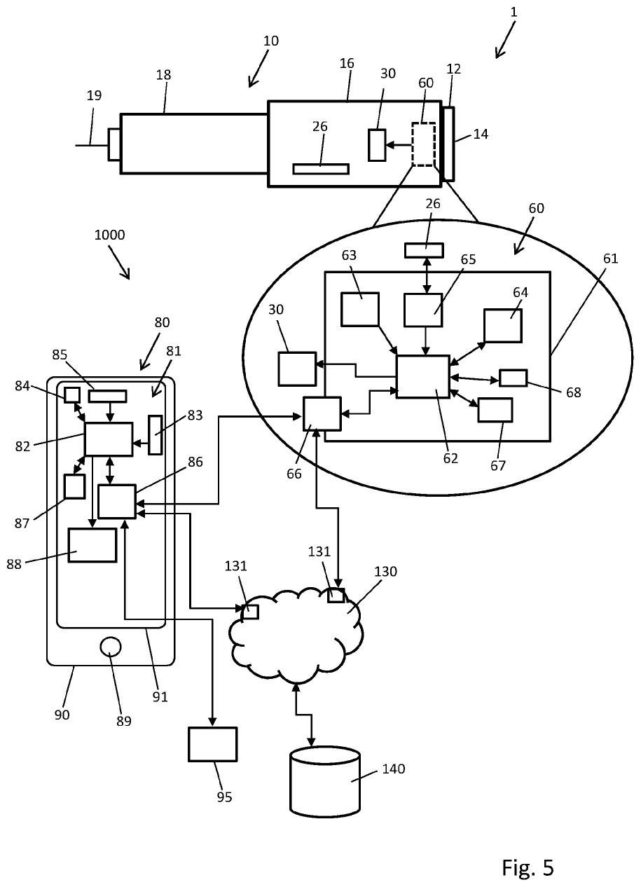

The injection device 10 further includes a drive mechanism in 11. The drive mechanism 11 includes a piston rod 24 of elongated shape and extending along the longitudinal direction of the housing 16. Inside the housing 16 there may be provided an inner body 15 serving as a support or mount of the drive mechanism 11. The piston rod 24 may be threadedly engaged with the inner body 15. A rotation of the piston rod 24 as induced by the drive mechanism 11 may thus lead to an advancing motion of the piston rod 24 relative to the inner body 15 and relative to the housing 16 so as to urge or to move the stopper 6 in distal direction 2 relative to the medicament container 5. This way, a dose of the medicament 8 can be expelled from the outlet of the medicament container 5. Typically, the seal 7 is pierceable by a double-tipped injection needle 19 as illustrated in FIGS. 4 and 5. The injection needle, e.g. including a threaded needle hub may be detachably or releasably connected to a distal end of the container housing.

The drive mechanism 11 typically includes a number sleeve 21 at least partially visible through a window 20 provided in the housing 16 of the injection device 10. Upon setting a dose the number sleeve 21 is subject to a rotating motion. Accordingly, an increasing sequence of dose indicating numbers may show up in the window 20 indicating the size of a dose currently set.

The drive mechanism 11 further includes a drive sleeve 22. The drive sleeve 22 is operatively connected or coupled with the piston rod when the drive mechanism 11 is in a dose dispensing or dose injection mode. Pushing the dose button 14 may initiate a rotation of the drive sleeve 22, which by activation of a clutch is operatively connected with the piston rod 24 so as to induce a dose dispensing rotation and a distally directed longitudinal advancing motion of the piston rod 24.

The piston rod 24 is provided with a pressure please 25 at its distal end. The pressure piece 25 is typically rotationally supported at the distal end of the piston rod 24. By way of the pressure piece 25 distally directed thrust as exerted by the piston rod 24 is transferred onto a proximal thrust receiving surface of the stopper 6. With some examples the drive mechanism 11 is provided with a mechanical energy storage, such as a spring. The mechanical energy storage may be biased during or upon setting of a dose. By depressing the dose button 14 mechanical energy stored in the mechanical energy storage may be released so as to provide a driving torque or driving force capable to advance the piston rod 24 in distal direction 2 for dispensing of a dose of the medicament 8. With other examples the drive mechanism 11 is void of a mechanical energy storage. Here, a force exerted by the user onto the dose button 14 is nearly entirely transferred into a driving force required for moving the piston rod 24 in distal direction.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

The drive mechanism 11 as described above is only exemplary for one of a plurality of differently configured drive mechanisms that are generally implementable in a disposable or reusable pen-injector. The drive mechanism as described above is explained in more detail e.g. in WO2004/078239A1, WO 2004/078240A1 or WO 2004/078241A1 the entirety of which being incorporated herein by reference. Further examples of drive mechanisms 11 to be implemented with the injection device 10 can be found in WO 2014/033195 A1 or WO 2014/033197 A1 the entirety of which being incorporated herein by reference. The drive mechanism as disclosed in WO 2014/033195 A1 is a reusable drive mechanism. The drive mechanism as disclosed in WO 2014/033197 A1 is an example of a disposable drive mechanism being void of a reset function.

In FIG. 4 numerous components of an authentication system 1000 as described below are schematically illustrated. The injection device 10 may be equipped with an electronic unit 60.

The electronic unit 60 typically includes an electronic circuit 61. The electronic circuit 61 includes a processor 62 and a battery 63. Moreover and with typical examples, the electronic unit 60 embedded into or integrated into the injection device 10 may also include a storage 64, a sensor 65, a communication interface 66, a proximity sensor 67, a signaling unit 68.

As indicated in FIG. 4, the electronic unit 60 may be arranged inside the dose dial 12. The dose button 14 may close a receptacle of the dose dial 12, which receptacle is sized to accommodate at least a portion of the electronic unit 60. As further illustrated in FIG. 4 the authentication system 1000 may include at least one mobile electronic device 80, 100, 120. The mobile electronic device 80 may be implemented as an external electronic device. It may include a smartphone. The smartphone 80 typically includes an input 89 and a display 91. Here, the input 89 may be integrated into the display 91, which is hence implemented as a touch sensitive display. With other examples the mobile electronic device 100 is implemented as a wearable electronic device. The wearable electronic device 100 may include a strap 112 or a wristband allowing to attach the mobile electronic device 100 e.g. to a wrist of a hand of a user. The mobile electronic device 100 may be implemented as a smartwatch.

Typically, the mobile electronic device includes a housing 110 accommodating an electronic circuit 101 equipped with a processor 102, a battery 103, a storage 104, a sensor 105, a communication interface 106, a proximity sensor 107 and a signaling unit 108. The housing 110 may be closed or covered by a display 111. With some examples the mobile electronic device 100 is attachable to a wrist of a hand or arm of a user 4. With other examples the mobile electronic device 100′ may be attached to another portion of a body of a user. As an example, the mobile electronic device 100′ may be implemented as a belt or may be integrated into a belt of a user.

The mobile electronic device 120 may be implemented as a wireless tag. The wireless tank 120 may include a housing 129. Typically, the wireless tag 120 includes an electronic circuit 121, e.g. including a printed circuit board. The electronic circuit 121 may include at least one of a processor 122, a battery 123, a sensor 125, a communication interface 126, a proximity sensor 127 and a signaling unit 128. The mobile electronic device 120 is typically carried along by the user 4.

With some examples the mobile electronic device 120 may be permanently attached to a personal item of a user, such as a key. With further examples the mobile electronic device 120 may be attached to the drug delivery device 1.

With the example of FIG. 5, the drug delivery device 1 and hence the injection device 10 is further equipped with a movable part 26. The movable part 26 may be implemented in the drive mechanism 11. A movable component of the drive mechanism 11 may form or constitute the movable part 26. A movement or position of the movable parts 26 of the drive mechanism 11 may be detected or tracked by the electronic unit 60 of the injection device 10.

A detectable position or orientation of the movable part 26 may be indicative of a current state or configuration of the injection device 10. A position of the movable part 26 along the longitudinal direction may be directly indicative of a residual amount of medicament 8 provided in the medicament container 5. By detecting or quantitatively measuring at least one of a position or orientation of the movable part 26, the electronic unit 60 may gather device specific information about the momentary status or configuration of the injection device. This way, the electronic unit 60 may be configured to collect and/or to gather data being indicative of an operational status or history of use of the injection device 10. The electronic unit 60 may collect data such as a dosing or dispensing history including information about an amount of medicament being dispensed at a particular date and/or time.

The injection device 10 may be further equipped with an electromechanical interlock 30. The electromechanical interlock 30 may be integrated into the drive mechanism 11. The interlock 30 may be operable by the electronic unit 60 of the injection device 10.

With the example of FIG. 5 the injection device 10 is equipped with the electronic unit 60. The electronic unit 60 includes e.g. a printed electronic circuit 61, a processor 62, a battery 63, a storage 64, at least one sensor 65, a communication interface 66, a proximity sensor 67 and/or a signaling unit 68.

The signaling unit 68 is configured to generate a user perceivable signal. It may include a speaker to generate an audible signal. It may include a display to visualize information to a user. It may include a blinking light, such as a LED. It may include numerous LEDs or light sources configured to generate light indication or light pulses of different color and/or duration. The signaling unit 68 may also include a buzzer or the like electromechanical unit to generate a vibration or some other haptically detectable signal.

The communication interface 66 is configured to communicate with another communication interface 86 of a mobile electronic device 80. Alternatively or additionally, the communication interface 66 is configured to communicate with a database 140 via a network 130. The communication interface 66 may access the database 140 and/or the network 130 by a particular access point 131. The communication between the communication interface 66 and the network 130 is typically implemented as a wireless communication link.

The mobile electronic device 80 may be implemented as a smartphone. It includes an electronic circuit 81, a processor 82, a battery 83, a storage 84, a sensor 85, a communication interface 86, a proximity sensor 87, a signaling unit 88, a user operable input 89, a housing 90 and a display 91. The mobile electronic device 80 may be configured to communicate with a satellite-based positioning system 95 so as to gather global or regional position information. Typically, the communication interface 86 of the mobile electronic device 80 is configured to communicate with the network 130 by at least one of several access points. It may communicate with the database 140 to gather or to receive requested data from the database 140.

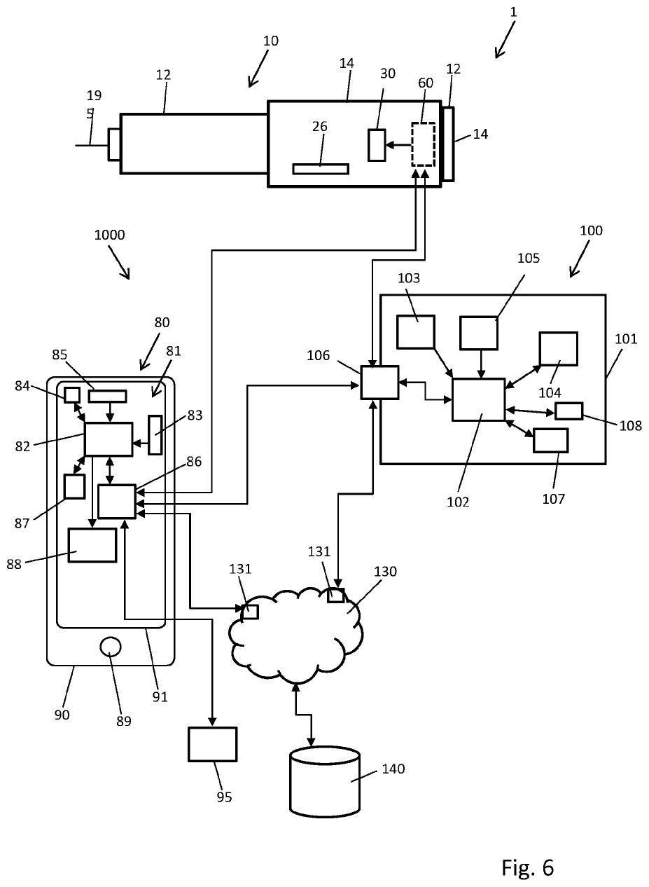

With the further example of an authentication system 1000 as illustrated in FIG. 6 there is provided not only one mobile electronic device 80 but also a further mobile electronic device 100, e.g. in form of a smartwatch or a fitness tracker. The mobile electronic device 100 also includes at least one of an electronic circuit 101, a processor 102, a battery 103, a storage 104, a sensor 105, a communication interface 106, a proximity sensor 107, and a signaling unit 108. These components may be all encapsulated in a housing 110. The signaling unit 108 may include a display 111. The signaling unit 108 may be implemented in the same or a similar way as the signaling unit 68 as described above in connection with the electronic unit 60.

The mobile electronic device 100 is operable to communicate with the network 130 and the database 140 via the communication interface 106. The mobile electronic device 100 may also wirelessly communicate with the electronic unit 60 of the drug delivery device 1 as well as with the communication interface 86 of the mobile electronic device 80. With further examples the mobile electronic device 100 may also directly or indirectly communicate with the satellite-based positioning system 95 so as to obtain geographic position data, e.g. for acquiring absolute or relative position data of the mobile electronic device 100.

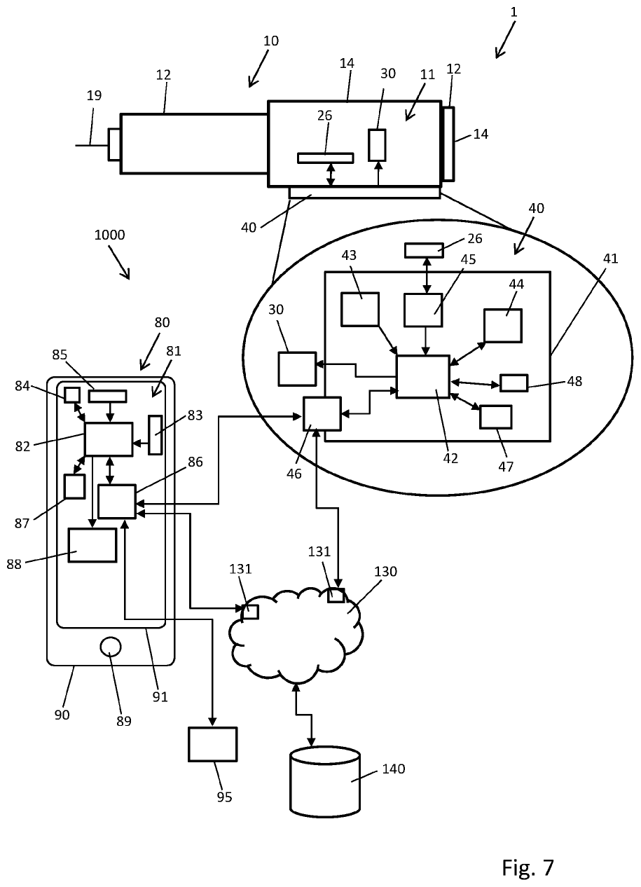

With the further example as illustrated in FIG. 7 the drug delivery device 1 may be void of an electronic unit 60 as described above. The injection device 10 and/or the drive mechanism 11 may be implemented all mechanically. Here, the injection device 10 may be equipped with a supplementary device 40. The supplementary device 40 typically includes a fastener 49 for detachably fastening or fixing of the supplementary device 40 to the housing 16 of the injection device 10.

The supplementary device 40 includes a signaling unit 48. The signaling unit 48 may include one of a light source, e.g. in form of a blinking light, a display, a speaker and a vibration generating unit. When implemented as a display or blinking light it may provide visible information or an visible alert to a user. Typically, the supplementary device 40 also includes at least one of an electronic circuit 41, a processor 42, a battery 43, a storage 44, a sensor 45, a communication interface 46 and a proximity sensor 47. With typical examples, the supplementary device 40 is operatively connected or coupled to the interlock 30 of the drive mechanism 11 when attached to the injection device 10.

Additionally or alternatively, the supplementary device 40, particularly its sensor 45 may be operatively connected or coupled to the movable part 26 of the drive mechanism 11. This way the supplementary device 40 is operable to acquire or to obtain information about the momentary status of the drive mechanism 11, which reflects in a position, orientation or movement of the movable part 26. Similarly and as described above in connection with the electronic unit 60 or with the mobile electronic device 100 the supplementary device 40 is operable to wirelessly communicate with a mobile electronic device 80, 100, 120.

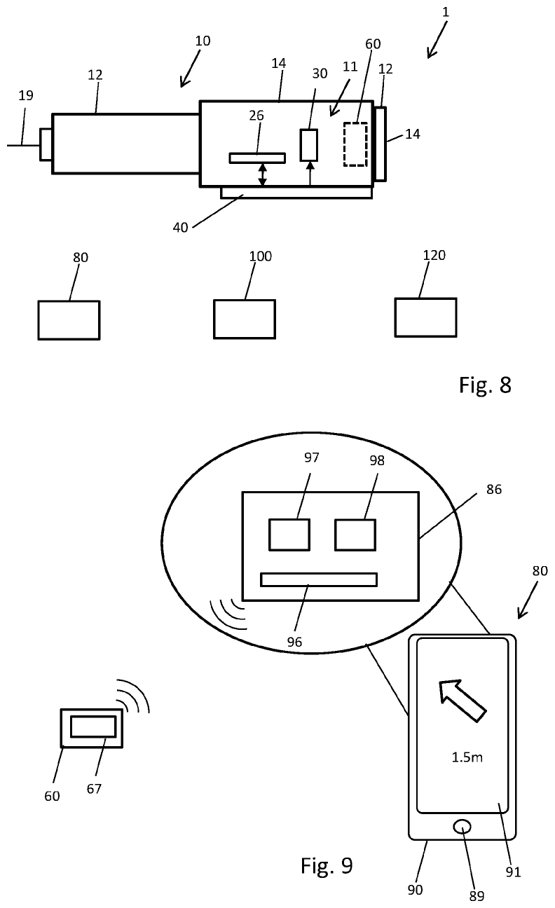

The supplementary device 40 may be also operable to directly communicate with a database 140 via the communication network 130. As further illustrated in FIG. 8, the supplementary device 40 and/or the electronic unit 60 of the injection device 10 may be operable to communicate with at least one or with several of the mobile electronic devices 80, 100, 120.

There may be established a wireless communication link between at least two of the electronic unit 60, the supplementary device 40, the mobile electronic device 80, the mobile electronic device 100 and mobile electronic device 120. Moreover, the mobile electronic devices 80, 100, 120 may communicate among each other.

There may be established a first wireless communication link between at least one of the electronic unit 60 and the supplementary device 40 with one of the mobile electronic devices 80, 100, 120. There may be established a further wireless communication link between any of the electronic unit 60, the supplementary device 40, the mobile electronic device 80, 100, 120 with the communication network 130 and/or with the database 140.

The sensor 45, 65, 85, 105, 125 may be implemented as one of an acceleration sensor, a rotation sensor, a position sensor, a distance sensor and a physiologic data capturing sensor of. Implementation of a physiologic data capturing sensor is typically provided with a wearable electronic device 80, 100, 120. By way of the sensor 45, 64, 85, 105, 125 motion data of the respective electronic circuit 41, 61, 81, 101, 121 can be acquired.

Respective motion data can be either stored in the associated storage 44, 64, 84, 104, 124 of the respective electronic circuit 61. The acquired motion data may be also communicated and/or transmitted via the communication interface 46, 66, 86, 108, 126 to another electronic circuit 41, 61, 81, 101, 121. Motion data of a user 4 may be also obtained by establishing a communication link with the database 140 via the network 130. Motion data as collected by any of the sensors 45, 65, 85, 105, 125 may be stored in the database 140 and can be made available to any other electronic circuit 41, 61, 81, 101, 121.

Moreover, at least a portion of the motion data can be acquired or collected by a first mobile electronic device 100, 120, e.g. carried along with the user 4.

A further portion of motion data can be acquired and collected by a sensor 44, 65 attached to or integrated into the injection device 10. Different portions of motion data may be combined and collected through a wireless communication link between respective mobile electronic devices 80, 100, 110 and at least one of the supplementary device 40 and the electronic unit 60. Any of the wireless communication interfaces 46, 66, 86, 106, 126 may be operable to transceive (to transmit and/or to receive) in a specific frequency range that follows a specific transmission protocol. The wireless communication interfaces may include multiple communications circuits, each transceiving and in a different frequency range and/or according to a different wireless transmission protocol.

At least one or several of the wireless communication interfaces 46, 66, 86, 106, 126 as described herein include at least one RFID-communication circuit (radio frequency identification). The wireless communication interface may include an NFC-circuit (near-field communication), e.g. an active NFC-circuit (preferably with associated power source, e.g. a battery such as a printable battery) or a passive NFC-circuit (preferably without a power source powering the circuit). The wireless communication interface may include at least one Bluetooth and/or BLE communication circuit (Bluetooth Low Energy). The wireless communication unit may include at least one WiFi-communication circuit (wireless fidelity, e.g. according to the IEEE 802.11 standard/protocol). Alternatively or additionally, the wireless communication unit may include a magnetometer/compass circuit to detect variation in magnetic fields, e.g. variations produced by a wireless communication circuit of a component of the drug delivery device.

With some examples at least one or several of the wireless communication interfaces 46, 66, 86, 106, 126 as described herein are implemented as UWB (ultra wideband) communication interfaces.

At least one of the wireless communication interfaces 46, 66, 86, 106, 126 is configured for generating UWB signals that enable communications between the another one of the wireless communication interfaces 46, 66, 86, 106, 126. The UWB signals include very short RF pulses (e.g., smaller than 1 ns) covering a large portion of the radio spectrum (e.g., bandwidth larger than 500 MHz or 20% of the center frequency, whichever is lower), at a very low energy level. The operating frequency is chosen in accordance with one or more national and federal regulations. For example, a frequency band with wide international acceptance is from about 6.5 GHz to about 8 GHz. In some implementations, the UWB signals include a UWB standard widely accepted and available in smartphones from large vendors such that standard smartphones can be used as the mobile electronic device 80.

In some implementations, the UWB communication includes a proprietary UWB protocol. The proprietary UWB protocol uses an encoding, which consists of a combination of time modulation, signal shape modulation, and amplitude modulation. A proprietary UWB device (e.g. USB dongle for a smartphone) can be used as a wireless tag 120. Transmission of UWB signals may be triggered by a user operation.

With some examples and as illustrated in FIG. 9 the wireless communication between at least one of the injection device 10 and the supplementary device 40 with at least one of the mobile electronic devices 80, 100, 120 provides a pager functionality. For instance when a communication link is established by using a UWB communication protocol a precise position tracking of the electronic unit 60 can be provided in principle. Hence, the position of at least one of the drug delivery device 10 and the supplementary device 40 can be precisely determined or measured within a precision of only a few centimeters or even millimeters.

In addition or alternative, the wireless communication link may also provide determination and/or quantitative measuring of at least one of a distance and a relative position between a first electronic circuit and a second electronic circuit, e.g. a distance and those or a relative position between the injection device 10 and one of the mobile electronic devices 80, 100, 120.

As indicated in FIG. 9 the mobile electronic device 80 includes a communication interface 86 equipped with an antenna 96, a distance unit 97 and a position detection unit 98. The antenna 96, e.g. implemented as a UWB antenna is operable to communicate with the electronic unit 60, alternatively with the electronic circuit 41 of the supplementary device 40. With some examples the antenna 96 and hence the communication interface 86 is operable to wirelessly communicate with the communication interface 66 and/or with a proximity sensor 67 of the electronic unit 60. The UWB antenna 96 is not limited to be implemented in the mobile electronic device 80. The antenna 96, the distance unit 97 and the position detection unit 98, configured to generate and/or to process UWB signals for a precise position and distance measurement between any two mobile electronic devices 80, 100, 120 and/or electronic circuit 41, 61 can be likewise implemented in any of the communication interfaces 46, 66, 106, 126.

The antenna 96 may be configured such that the time characteristics of the UWB signals are constant over the frequency spectrum, resulting in minimal pulse distortion. The antenna 96 may exhibit a rather flat frequency spectrum, resulting in wide pulses with minimal resonant distortion. The antenna 96 may be integrated into any one of the communication interfaces 46, 66, 86, 106, 126, typically near a surface of a respective housing to have minimal attenuation of the signal. Possible implementations of the antenna 96 are the integration of a chip antenna, or the integration of a conductive layer acting as the antenna 96 on one of a plastic component of any one of the injection device 10, supplementary device 40, or any one of the mobile electronic device 80, 100, 120. A counterpart communication interface 46, 66, 86, 106, 126 operable to communicate with the antenna 96 may be configured to constantly or intermittently listen for incoming data packets transmitted by the antenna 96.

The distance unit 97 operatively coupled to the antenna 96 may be capable to derive or to quantitatively measure a distance between the electronic unit 60 and the communication interface 86, hence between the electronic unit 60 and the device 80 on the basis of at least one of signal dispersion, time of flight measurement, dynamic or static triangulation or on the basis of any further generally available distance or position measurement scheme being compatible with the respective wireless communication protocol.

The antenna 96 may be implemented as an array antenna being capable to determine a direction from which a signal or a response signal is received from the electronic unit 60. By way of the wireless communication link between the mobile electronic device 80 and one of the electronic unit 60 and the supplementary device 40 not only a distance, such as 1.5 m but also a direction and hence a relative position between the device 10, 40 and 80 can be provided in form of a visible symbol, e.g. on the display 91 of the mobile electronic device 80, thus assisting a user 4 to find or to retrieve the electronic unit 60 in case the respective device should get lost or in case the user should be currently unaware of a place where the injection device 10 can be found.

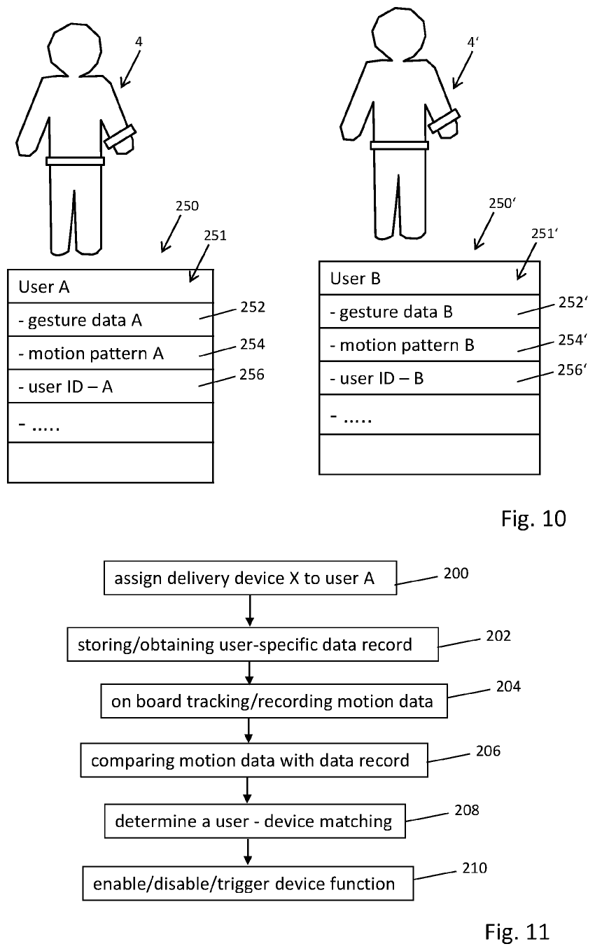

In FIG. 10 a data record 250 of a first person 4 and another data record 250′ of another person 4′ are illustrated. The data records 250, 250′ include motion data 251, 251′. Motion data 251 includes at least one of gesture data 252 and a motion pattern 254 of the user 4. Gesture data 252 characteristic of particular movements or gestures conducted or executed by a user 4, e.g. the way the user walks or the way the user picks up a device.

The motion pattern 254 may be characteristic or indicative of a motion profile of the respective user 4. The motion pattern 254 may contain information about the habits of the user for. E.g. the motion pattern 254 may indicate that a particular user A is located in his home environment at particular days a week and stays there during a specific time interval, e.g. during night times.

The motion pattern 254 may contain a typical movement profile and may provide a mapping of the position of the user over time. Optionally, the data record 250 may also include a user identification 256. The user identification 256 may contain a unique identifier of the user. The data record 250 may be subject to a permanent update as the user 4 is equipped with a mobile electronic device 80, 100, 120 being incapable to capture and/or to acquire respective motion data or movement data over time.

The data record 250 includes motion data 251 and be stored in any of the above-mentioned storages 44, 64, 84, 104, 124. It may be made available upon request, e.g. when the authentication system 1000 conducts a comparison of actually or newly acquired motion data 261 with previously stored motion data 251 in order to determine or to derive a data matching index 270.

As further illustrated in FIG. 10 another user B includes another data record 250′. Also this data record 250′ may be structured the same way as the data record 250. The data record 250′ also includes gesture data 252′, a motion pattern 254′ and optionally also a user identification 256′. The motion data 251 is characteristic for a first user 4 while the motion data 251′ is characteristic for another user 4′.

As further illustrated in FIG. 11 and when deploying at least one of a drug delivery device 1 and a supplementary device 40 to a particular user 4 the delivery device 1 is assigned to the user A in step 200. Upon deploying the authentication system 1000 the motion data 251 of user A is either acquired over time or is obtained from a database 140. In step 202 the respective user specific data record 250 is stored in one of the storages 44, 64, 84, 104, 124 and can be made available upon request.

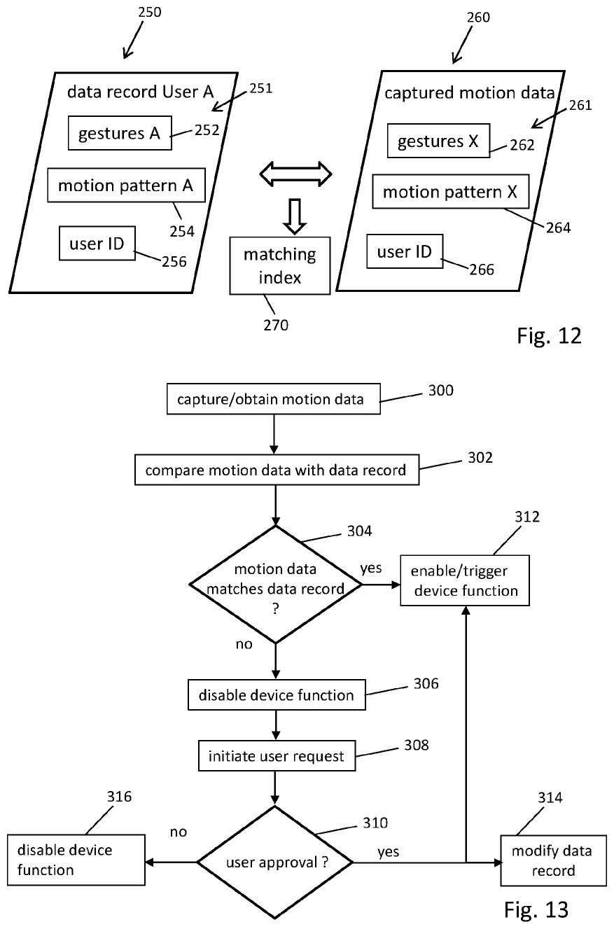

In a subsequent step 204 and when the user 4 intends to use a drug delivery device 1 actual or recent motion data 261 as illustrated in FIG. 12 is acquired from or derived from motions of the user 4. Acquiring of actual motion data 261 is conducted e.g. by at least one of the sensors 45, 65, 85, 105, 125 as described above or respective motion data 261 may be acquired or obtained via wireless communication as provided by one of the above-mentioned communication interfaces 46, 66, 86, 106, 126, e.g. either directly or indirectly from the database 140.

In a subsequent step 206 the actually required motion data 261 is compared with previously stored motion data 251 of the stored data record 250 which has been assigned to the user 4.

Hence, the motion data 261 recently acquired is compared with previously stored motion data 251. Here, stored gesture data 252 can be compared with actually recorded or acquired gesture data 262. Additionally or alternatively, a motion pattern or movement profile 264, which is actually acquired can be compared with a previously stored motion pattern 254.

In step 208 the comparison leads to the generation of a data matching index 270 and thus to a determination if the captured motion data of the user 4 matches with previously stored motion data of the user that has been assigned with the respective drug delivery device 1. If there is a high degree of matching between the acquired motion data 261 and the stored motion data 251 a particular device function may be then enabled, disabled, trigger or terminated. Generally, there is provided a variety of device functions that may be enabled, disabled, triggered or terminated upon determination of a user being authorized to use the drug delivery device.

With a high degree of a data matching e.g. setting of a dose of the injection device may be enabled, an interlock may be disabled, a user guidance routine may be triggered and/or a standby mode of the injection device may be terminated.

Concurrently, and when for instance there is revealed only a low degree of matching of acquired motion data 261 with previously stored motion data 251 an interlock may be activated or enabled, a separate authentication routine may be triggered by way of which the user may have to manually authorize himself through another authorization scheme of the injection device or the mobile electronic device. With further scenarios and with a low degree of data matching a standby mode of the injection device 10 and/or of the supplementary device 40 may be maintained.

With some examples an optional user ID 266 may be acquired from a user and may be compared with a stored user ID 256 of the user's data record. Comparison of user IDs may require that the user manually authenticates with one of the mobile electronic device 80, 100, 120, e.g. by entering a code, by a fingerprint recognition or by a camera-based face recognition procedure.

Additionally, the captured motion data may also include physiologic data of the user 4. Respective physiologic data may be also stored in the data record 250. Physiologic data may include at least one of a heartbeat rate, a breathing frequency, a blood pressure profile, an oxygen saturation or other measurable physiological parameters of the user that may be principally stored in the data record 250. Measuring such physiologic data by a sensor 85, 105, 125 of a mobile electronic device 80, 100, 120 may enable to conduct a comparison of stored physiologic data with actually measured physiologic data thus allowing to identify a user of the mobile electronic device 80, 100, 120.

In FIG. 13 there is described an example of a method of authenticating a user to use a drug delivery device. In a step 300 motion data of the user 4 is captured or obtained either by making use of a mobile electronic device 80, 100, 120 or by an electronic circuit 41 of the supplementary device 40 and/or by an electronic circuit 61 of an electronic unit 60 integrated into the injection device 10. The captured motion data 261 is then compared with previously stored motion data 251 of a user 4 that is authorized to use the drug delivery device 1. This comparison is conducted in step 302. In a subsequent step 304 the comparison is evaluated and the data matching index 270 is generated. In step 304, the data matching index being indicative of a matching of previously stored motion data and actually captured motion data 261 is evaluated and analyzed.

With a low degree of matching the method continues with step 306 and a particular device function is e.g. disabled. As a subsequent step 380 the method of authenticating authorizing may then continue to initiate a user request. You, the user is given a further possibility to authenticate in front of the drug delivery device. In step 308 the user may be prompted to conduct a manually authorization procedure, e.g. by entering a code in one of the mobile electronic devices 80, 100, 120. If the user correctly enter a code and thus authorizes appropriately the method continues with step 312, in which a respective device function is enabled or triggered.

Optionally, or additionally and particularly in cases wherein the data matching index may not unequivocally lead to an approval or disapproval of enabling or disabling of the device function the captured motion data may be used to modify the stored data record in step 314. This way, the method is of self-learning type and may automatically adapt to varying user habits. When in step 310 the user fails to authenticate with the injection device 10 or with any of the mobile electronic devices 80, 100, 120 the device function may remain disabled in step 316.

Generally, and if in step 304 the captured motion data 261 matches the previously stored motion data 251 of the user-specific data record 250 the method directly continues with step 312. Accordingly, and when the acquired or captured motion data 261 matches the previously stored motion data 251 a respective device function is enabled or triggered without requesting any further authentication of the user 4. Here, the user 4 may not even be aware, that he unlocks or activates a device function simply by tracking or recording one of his gestures and/or his motion pattern. In this way, a user authentication can be simplified smoothly integrated into the user's daily life and may no longer require any active interaction with the user 4.

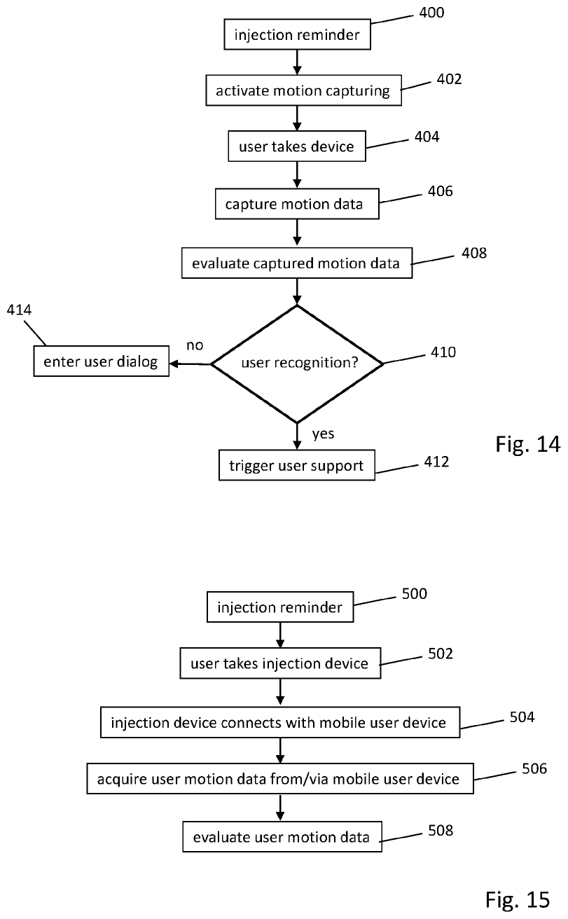

In FIG. 14 another flowchart of a method to authenticate a user to use a drug delivery device is illustrated. Here, in a first step 400, a user or may be reminded by a software routine of one of the mobile electronic devices 80, 100, 120 or of the supplementary device 40 to conduct a drug delivery procedure, e.g. an injection procedure. In a subsequent step 402 to the motion capturing or acquisition of motion data may be activated with at least one of the electronic circuit 41, the electronic unit 60 and/or one of the mobile electronic devices 80, 100, 120.

In a subsequent step 404 the user 4 for may for instance take the drug delivery device 1. Here, a motion sensor 65 either implemented in the electronic unit 60 or another motion detector or sensor 45 integrated into the supplementary device 40 and attached to the drug delivery device 1 starts capturing or acquiring respective motion data in step 406. In step 408 the motion data thus obtained is evaluated and/or analyzed. Here, in step 408 the actually acquired motion data 261 is compared with previously stored motion data 251 of user being authorized to use the drug delivery device 1.

If in a subsequent step 410 the user is recognized as a user being authenticated or authorized to use the drug delivery device 1 a user support routine may be triggered, either on board of the supplementary device 40 or by the electronic unit 60 of the drug delivery device 1. For instance, the drug delivery device and/or the supplementary device 40 may be switched from a standby mode into an active mode. Optionally and by way of a communication link with at least one of the mobile electronic devices 80, 100, 120 a user assisting routine may be triggered so as to assist the user to execute the drug delivery procedure.

If in step 410 a user recognition fails, e.g. due to a low degree of matching between the acquired motion data 261 and the stored motion data 251 the procedure may continue to enter into a user dialog in step 414. A particular device function may be disabled and the user 4 may be prompted to authenticate himself, e.g. via one of the mobile electronic devices 80, 100, 120 or by interaction with the supplementary device 40. Here, the user 4 may be prompted to enter a code or to conduct any other authentication routine, such as fingerprint detection or face recognition.

In the flowchart of FIG. 15 another way of implementing the method of authenticating a user to use a drug delivery device 1 is schematically illustrated. In a first step 500 the user is reminded to conduct an injection procedure and to follow a pre-defined injection schedule. Here, in step 502 the user takes the injection device 10. In a subsequent step the injection device 1, in particular its electronic unit 60 connects with a mobile electronic device 80, 100, 120 of the user 4. Motion data 251, 261 of the user, e.g. a motion profile or motion pattern 254, 264 of the user which has been previously or just recently collected and acquired by the mobile electronic device 80, 100, 120 is provided to the electronic unit 60 of the injection device 10.

This may be provided by a wireless communication link between the electronic unit 60 and at least one of the mobile electronic devices 80, 100, 120. Then, the motion data 251, 261 as previously or recently acquired by at least one of the mobile electronic devices 80, 100, 120 is provided to the electronic unit 60 of the drug delivery device 1 and is then further evaluated in step 508 in a similar way as described above. The electronic unit 60, in particular its processor and 62 may then at least one of enable, disable, trigger or terminate execution of at least one function of the drug delivery device. With this implementation, the electronic unit 60 of the injection device 10 may be void of an sensor 65 or proximity sensor 67. Respective motion data by way of which a user 4 of the injection device can be characterized may be obtained through a wireless communication link between the electronic unit 60 and any one of the mobile electronic devices 80, 100, 120.

In still other implementations of the method, the computer program and the authentication system 1000 it is also conceivable, that evaluation and/or analysis of motion data is not conducted on board by the supplementary device 40 or by the electronic unit 60 of the injection device but. Rather, data analysis or data evaluation may be conducted by at least one of the database 140 or by at least one of the mobile electronic devices 80, 100, 120. In this way, the computational power required for the data analysis or data evaluation can be provided remote from the drug delivery device 1 thus allowing to save energy with the drug delivery device 1.

With some examples the mobile electronic device 80, 100, 120 may be paired with the electronic unit 60 and/or with the supplementary device 40. This way, only one of a plurality of mobile electronic devices 80, 100, 120 is given the possibility to communicate with the supplementary device 40 or with the electronic unit 60. Accordingly, only one of a plurality of mobile electronic device 80, 100, 120 may be authorized to submit any commands to the supplementary device 40 or to the electronic unit 60 of the drug delivery device 1. In circumstances, wherein such an electronic device 80, 100, 120, which is paired or authenticated communicate with the electronic unit 60 or with the supplementary device is used by another user not being authorized or authenticated to use the respective drug delivery device the acquisition and evaluation or analysis of motion data can be effectively used to prevent unauthorized use of the drug delivery device. This way, patient safety and compliance with a prescribed or intended use of the drug delivery device 1 or injection device 10 can be improved with a minimum impact on user interaction.

### REFERENCE NUMBERS

1 drug delivery device
2 distal direction
3 proximal direction
4 user
5 medicament container
6 stopper
7 seal
8 medicament
10 injection device
11 drive mechanism
12 dose dial
14 dose button
15 inner body
16 housing
17 protective cap
18 container housing
19 injection needle
20 window
21 number sleeve
22 drive sleeve
24 piston rod
25 pressure piece
26 movable part
30 interlock
40 supplementary device
41 electronic circuit
42 processor
43 battery
44 storage
45 sensor
46 communication interface
47 proximity sensor
48 signaling unit
49 fastener
50 housing
51 display
60 electronic unit
61 electronic circuit
62 processor
63 battery
64 storage
65 sensor
66 communication interface
67 proximity sensor
68 signaling unit
80 mobile electronic device
81 electronic circuit
82 processor
83 battery
84 storage
85 sensor
86 communication interface
87 proximity sensor
88 signaling unit
89 input
90 housing
91 display
95 satellite-based positioning system
96 antenna
97 distance unit
98 position detection unit
100 mobile electronic device
101 electronic circuit
102 processor
103 battery
104 storage
105 sensor
106 communication interface
107 proximity sensor
108 signaling unit
110 housing
111 display
112 strap
120 mobile electronic device
121 electronic circuit
122 processor
123 battery
125 sensor
126 communication interface
127 proximity sensor
128 signaling unit
129 housing
130 network
131 access point
140 database
250 data record
251 motion data
252 gesture data
254 motion pattern
256 user identification
260 data record
261 motion data
262 gesture data
264 motion pattern
266 user identification
270 matching index
1000 authentication system

1-19. (canceled)

20. A method of authenticating a user for using a drug delivery device, the method comprising:
acquiring motion data of the user;
comparing acquired motion data with stored motion data of a user-specific data record and generating a data matching index being indicative of a degree of matching between the acquired motion data and the stored motion data; and
enabling, disabling, triggering, or terminating an execution of a function of the drug delivery device based on the data matching index.

21. The method according to claim 20, wherein the motion data of the user is acquired by an electronic circuit comprising at least one of a communication interface and a sensor.

22. The method according to claim 21, wherein at least a portion of the acquired motion data is acquired by the sensor.

23. The method of claim 22, wherein the sensor comprises at least one of an acceleration sensor, a rotation sensor, a position sensor, a distance sensor, and a physiologic data capturing sensor.

24. The method according to claim 21, wherein at least a portion of the acquired motion data is acquired by the communication interface through a wireless communication with at least one of:
a satellite-based positioning system,
an access point of a communication network, and
a counterpart communication interface of at least one of:
a supplementary device attachable to the drug delivery device,
an electronic unit of the drug delivery device, and
a mobile electronic device).

25. The method according to claim 20, wherein the acquired motion data comprises at least one of:
gesture data being indicative of characteristic gestures of the user, and
a motion pattern being indicative of at least one of a position and a motion profile of the user at a specific point of time or during a specific time interval.

26. The method according to claim 20, wherein the user-specific data record is obtained by at least one of:
deriving and storing the stored motion data from previously acquired motion data of the user; and
requesting and receiving at least one of a user-specific motion pattern and user-specific gesture data from a database.

27. The method according to claim 20, wherein the execution of the function of the drug delivery device is enabled or triggered when the data matching index is above an upper data matching threshold.

28. The method according to claim 27, wherein the execution of the function of the drug delivery device is disabled or terminated when the data matching index is below a lower data matching threshold.

29. The method according to claim 28, further comprising:
entering into a dialogue with the user;
generating and outputting a user request when the data matching index is below the upper data matching threshold and above the lower data matching threshold; and
enabling or triggering the function of the drug delivery device in response to the dialogue with the user or in response to a reaction of the user to the user request.

30. An authentication system for authenticating a user for using a drug delivery device, the authentication system comprising:
an electronic circuit integrated into or attachable to the drug delivery device, the electronic circuit being operable to enable, disable, trigger, or terminate an execution of a function of the drug delivery device;
at least one of a sensor and a communication interface operable to acquire motion data of the user;
a storage operable to store motion data of a user-specific data record; and
at least one processor operable to compare acquired motion data to stored motion data and operable to generate a data matching index being indicative of a degree of matching between the acquired motion data and the stored motion data, wherein the data matching index is configured to effectuate at least one of an enabling, disabling, triggering, or terminating the execution of the function of the drug delivery device.

31. The authentication system according to claim 30, wherein the electronic circuit is part of a supplementary device attachable to the drug delivery device, or wherein the electronic circuit is part of an electronic unit integrated into the drug delivery device.

32. The authentication system according to claim 30, further comprising a mobile electronic device comprising at least one of the sensor and the communication interface for acquiring at least a portion of the motion data of the user.

33. The authentication system according to claim 32, wherein the electronic circuit comprises a circuit communication interface configured to wirelessly communicate with the communication interface of the mobile electronic device.

34. The authentication system according to claim 33, wherein the circuit communication interface is operable to receive at least the portion of the motion data acquired by the mobile electronic device.

35. The authentication system according to claim 30, wherein the mobile electronic device comprises at least one of an external electronic device, a wearable electronic device, and a wireless tag.

36. The authentication system according to claim 30, wherein the at least one processor is operable to enable, disable, trigger, or terminate the execution of the function of the drug delivery device depending on the data matching index.

37. The authentication system according to claim 30, wherein at least a portion of the acquired motion data is acquired by the sensor, and wherein the sensor comprises at least one of an acceleration sensor, a rotation sensor, a position sensor, a distance sensor, and a physiologic data capturing sensor.

38. A computer program that is executable by at least one processor of an authentication system for authenticating a user for using a drug delivery device, wherein the authentication system comprises:
an electronic circuit integrated into or attachable to the drug delivery device, the electronic circuit being operable to enable, disable, trigger, or terminate an execution of a function of the drug delivery device;
at least one of a sensor and a communication interface operable to acquire motion data of the user;
a storage operable to store motion data of a user-specific data record; and
at least one processor operable to compare acquired motion data with stored motion data and operable to generate a data matching index being indicative of a degree of matching between the acquired motion data and the stored motion data, wherein the data matching index is configured to effectuate at least one of an enabling, disabling, triggering, or terminating the execution of the function of the drug delivery device; and
wherein the computer program product comprises computer readable instructions that, when the computer program product is executed by the at least one processor of the authentication system, causes the at least one processor to:
acquire the motion data of the user;
compare the acquired motion data to the stored motion data of the user-specific data record;
generate the data matching index ; and
enable, disable, trigger, or terminate the execution of the function of the drug delivery device based on the data matching index.

39. A set configured for use with or in an authentication system, the authentication system comprising:
an electronic circuit integrated into or attachable to the drug delivery device, the electronic circuit being operable to enable, disable, trigger, or terminate an execution of a function of the drug delivery device;
at least one of a sensor and a communication interface operable to acquire motion data of the user;
a storage operable to store motion data of a user-specific data record; and
at least one processor operable to compare acquired motion data to stored motion data and operable to generate a data matching index being indicative of a degree of matching between the acquired motion data and the stored motion data, wherein the data matching index is configured to effectuate at least one of an enabling, disabling, triggering, or terminating the execution of the function of the drug delivery device;
the set comprising one or more of:
the drug delivery device, wherein the drug delivery device is provided with the electronic circuit of the authentication system,
a supplementary device attachable to the drug delivery device and provided with the electronic circuit of the authentication system, and
a mobile electronic device comprising at least one of the sensor and the communication interface of the authentication system.
